(19) United States
(12) Patent Application Publication
(10) Pub. No.: US 2022/0219165 A1
(43) Pub. Date: Jul. 14, 2022

(54) METHOD OF MANUFACTURING A MICROFLUIDIC ARRANGEMENT, METHOD OF OPERATING A MICROFLUIDIC ARRANGEMENT, APPARATUS FOR MANUFACTURING A MICROFLUIDIC ARRANGEMENT

(71) Applicant: OXFORD UNIVERSITY INNOVATION LIMITED, Oxford (GB)

(72) Inventors: Edmond WALSH, Oxford, Oxfordshire (GB); Peter Richard COOK, Oxford, Oxfordshire (GB)

(21) Appl. No.: 17/607,227

(22) PCT Filed: Jun. 8, 2020
(86) PCT No.: PCT/GB2020/051383

§ 371 Date: Oct. 28, 2021

(30) Foreign Application Priority Data
Jun. 21, 2019 (GB) 1908926.7

(51) Int. Cl. B01L 3/00

(52) U.S. Cl. CPC B01L 3/502707; B01L 3/50273; B01L 2300/089; B01L 2200/0673; B01L 2300/0816; B01L 2200/12

(57) ABSTRACT

Methods and apparatus for manufacturing and operating a microfluidic arrangement are disclosed. In one arrangement, a continuous body of a first liquid is provided in direct contact with a first substrate. A second liquid is provided in direct contact with the continuous body of first liquid and covering the continuous body of first liquid, the second liquid being immiscible with the first liquid. A separation fluid, immiscible with the first liquid, is propelled through at least the first liquid and into contact with the first substrate over all of a selected region on the surface of the first substrate, thereby displacing first liquid that was initially in contact with the selected region away from the selected region without any solid member contacting the selected region directly and without any solid member contacting the selected region via a globule of liquid held at a tip of the solid member, the selected region being such that one or more walls of second liquid are formed that modify a shape of the continuous body of first liquid.

The invention relates to creating and operating a microfluidic arrangement and is particularly applicable to the case where the microfluidic arrangement is to be used for scientific experiments on biological matter such as living cells or other biological material.

Microwell plates are widely used for studies involving biological material. Miniaturisation of the wells allows large numbers of wells to be provided in the same plate. For example, plates having more than 1000 wells, each having a volume in the region of tens of nanolitres, are known. Miniaturisation is difficult due to the intrinsic need to provide solid walls that separate the wells from each other. The thickness of these walls reduces the surface area available for the wells.

Microwell plates also lack flexibility because the size of the wells and the number of wells per plate is fixed. Furthermore, biological and chemical compatibility can be limited by the need to use a material that can form the structures corresponding to the wells in an efficient manner. For example, for high density plates it may be necessary to use a material such as polydimethylsiloxane (PDMS), but untreated PDMS has poor biological and chemical compatibility because it leaches toxin and reacts with organic solvents.

The provision of flowing systems is also important for biological applications (e.g. where fresh nutrients must be supplied and waste material removed). Implementation of such systems at the microscale has proven a challenge for live cell based assays. Such systems regularly suffer from air bubbles and difficulties extracting cells. Many systems are made from PDMS, which has the problems mentioned above.

EP 1 527 888 A2 discloses an alternative approach in which ink jet printing is used to form an array of closely spaced droplets of growth medium for culture and analysis of biological material. This approach provides more flexibility than a traditional microwell plate but requires sophisticated equipment to perform the printing. Additionally, it is time consuming to add further material to the droplets after the droplets have been formed and there is significant footprint not wetted by the resultant sessile drops as they do not tessellate.

A further challenge in working with microfluidic arrangements is that implementation of high quality flow controlling elements such as valves can be difficult and/or expensive due to the small sizes involved.

It is an object of the invention to provide alternative ways of creating and/or operating microfluidic arrangements.

According to an aspect of the invention, there is provided a method of manufacturing a microfluidic arrangement, comprising: providing a continuous body of a first liquid in direct contact with a first substrate; providing a second liquid in direct contact with the continuous body of first liquid and covering the continuous body of first liquid, the second liquid being immiscible with the first liquid; and propelling a separation fluid, immiscible with the first liquid, through at least the first liquid and into contact with the first substrate over all of a selected region on the surface of the first substrate, thereby displacing first liquid that was initially in contact with the selected region away from the selected region without any solid member contacting the selected region directly and without any solid member contacting the selected region via a globule of liquid held at a tip of the solid member, the selected region being such that one or more walls of second liquid are formed that modify a shape of the continuous body of first liquid.

The method allows a microfluidic arrangement containing one or more liquid walls to be formed flexibly on a substrate without any mechanical or chemical structures being provided beforehand to define the geometry of the walls. The shapes and sizes of the walls are defined by the geometry of the selected region, which defines the area on the first substrate where the first liquid has been displaced. The second liquid fills the space left by the first liquid and prevents flow of the first liquid through the region occupied by the new liquid wall. The one or more walls may be arranged to define flow conduits and/or may completely isolate sub-bodies of the first liquid from other sub-bodies of the first liquid. As described below, the choice of the selected region is relatively unrestricted. It is possible to create extremely narrow and/or closely spaced flow conduits or sub-bodies, for example of the order of 100 microns or smaller, which would be difficult or impossible to create at reasonable cost and/or time, without surface modification/treatment, using standard manufacturing techniques (such as microwell plate manufacturing techniques). The liquid walls of embodiments of the present disclosure typically have a thickness of 70-120 microns (and can be created at thicknesses down to around 1 micron), which allows more than 90% of the surface area of the microfluidic arrangement to be available for containing liquids to be manipulated. Furthermore, there are no solid walls to interfere with adding further liquid to the microfluidic arrangement, and gas bubbles (a difficulty in classical microfluidics) are easily removed by buoyancy forces, either passively or manually (assisted by the intrinsically improved accessibility provided by the absence of solid walls). The approach is particularly suited to efficiently providing microfluidic arrangements suitable for providing a constant or pseudo-constant flow of liquid containing nutrients past or through chambers containing biological cells.

In comparison with arrays of droplets deposited by ink jet printing or the like, the method avoids the need for sophisticated printing equipment and can achieve higher space filling efficiency (because the shapes of features of the microfluidic arrangement do not need to be circular).

In an embodiment, each of the one or more walls of second liquid is pinned in a static configuration by interfacial forces. The pinning is such that each of the walls of second liquid has a wall footprint representing an area of contact between the second liquid and the first substrate that remains constant. In an embodiment, an outline of the wall footprint of at least one of the walls comprises at least one straight line segment. Straight line segments can be formed efficiently by an appropriate scanning action of a distal tip. Straight line segments allow higher space filling efficiency in comparison with geometries defined, for example, by circular or elliptical bodies of liquid. In an embodiment, the outline of the wall footprint of at least one of the walls comprises at least two straight line segments that are non-parallel to each other, for example perpendicular to each other. The straight line segments may form portions of square, rectangular or other tessellating shapes for example.

In an embodiment, the one or more walls define at least one open-ended flow conduit. In an embodiment, the one or more walls further define a microfluidic arrangement connected to the open-ended flow conduit at an end of the open-ended flow conduit opposite to the open end, the microfluidic arrangement and open-ended flow conduit being configured such that the open end acts as a passive check valve separating the microfluidic arrangement from a macroscopic sink volume. This approach provides a simple and effective way of implementing check valve functionality in microfluidic arrangements.

In an embodiment, the separation fluid is propelled onto the selected region on the first substrate by pumping the separation fluid from a distal tip of an injection member while moving the distal tip relative to the first substrate. This approach can be implemented using relatively simple hardware in a cost-effective and reliable manner. Alternative approaches which involve contact of a solid member with the selected region (e.g. using scraping of the solid member along the selected region), require a degree of clearance to be provided in a mounting arrangement of the solid member to allow for movement of the solid member perpendicular to the surface of the first substrate (i.e. in the z-direction). In comparison to such approaches, the present approach can provide higher resolution because no movement of the injection member perpendicular to the surface of the first substrate (z-direction) is required. The injection member can thus be clamped rigidly without any clearance (with respect to the clamping arrangement) in directions parallel to the surface of the first substrate (x-y directions), which improves positioning accuracy. Positioning accuracy will be limited only by the accuracy of the mechanism used to move the injection member over the first substrate. The removal of the need for contact between the injection member and the first substrate also means that the approach is less sensitive to errors caused by height variations in the surface of the first substrate and/or does not need to compensate for such height variations. The absence of required z-direction movement also improves speed relative to alternative approaches which involve contact of a solid member with the selected region (where time-consuming z-direction movement is required). The absence of contact also reduces maintenance requirements, for example by avoiding accumulation of molecules over time on a contacting member, which would lead to cleaning or replacement operations being required. Furthermore, the avoidance of such accumulation reduces or removes the risk of cross-contamination between different regions of the microfluidic arrangement caused by the contacting member.

The use of a separation fluid propelled onto the surface of the substrate also provides enhanced flexibility relative to alternative approaches which involve contact of a solid member with the selected region. Where a solid member is used to cut through the first liquid along a path corresponding to a selected region, the width of the cut is defined by the fixed size and shape of the solid member. If a different sized cut is required it would be necessary to replace the solid member with a different solid member. Furthermore, manufacturing errors in the solid member will lead to corresponding errors in the width of cut. In the present approach, in contrast, the width of the cut can be varied by altering the way the separation fluid is propelled onto the surface, for example by altering the velocity of the separation fluid, the distance between the injection member and the surface, the time the injection member resides in a certain position or the speed at which the injection member is scanned over the surface, or the diameter of the jet of separation fluid. Manufacturing errors in the injection member will not cause corresponding errors in the width of cut, and moreover tubes which are commonly, and cheaply, available with high tolerance, e.g. hollow stainless steel needles, can be used as the injection member and/or custom needles may be used.

It has been observed that alternative approaches which involve contact of a solid member with the selected region can have a significant risk of producing walls that have unwanted breaks (thereby undesirably allowing the first liquid to flow through a region where it was intended that the wall would prevent such a flow). For example, it has been observed that in arrays of sub-bodies containing cell-culture medium produced using the alternative approach a small subset of the sub-bodies are found to be connected together. Without wishing to be bound by theory, it is thought that these unwanted connections may result from proteins or other material in the cell-culture medium attaching to the solid member while it is being moved along the selected region and disrupting the process of cutting of the first liquid into the sub-bodies by the solid member. This mechanism does not arise with the non-contact methods proposed herein and, indeed, unwanted incomplete separation of sub-bodies has not been observed using otherwise similar conditions and cell-culture medium.

It has also been observed that in alternative approaches which involve contact of a solid member with the selected region, debris (e.g. vesicles, protein aggregates in cell-culture medium) can accumulate on the solid member while it is being used to cut the first liquid along a path corresponding to a selected region. This suggests that the cutting process may remove materials from the first liquid and thereby undesirably modify or disrupt the composition of the first liquid. Furthermore, the contact from the solid member can introduce defects or cuts along the selected region, which can also attract debris such as vesicles or lumps of protein. Such modifications or disruptions will be lower or negligible using the non-contact approach of the present disclosure.

In an embodiment, the distal tip is moved through both of the second liquid and the first liquid while propelling the separation fluid onto the selected region on the first substrate, for at least a portion of the selected region. In embodiments of this type, the movement of the distal tip assists with displacing the first liquid away from the volume adjacent to the selected region, thereby improving efficiency. In an embodiment, at least a portion of the distal tip of the injection member is configured to be more easily wetted by the second liquid than the first liquid. This facilitates efficient displacement of the first liquid by the second liquid by promoting efficient dragging of the second liquid through the first liquid in the wake of the distal tip. The dividing process can thereby be performed more reliably and/or at higher speed.

In an embodiment, the separation fluid comprises a portion of the second liquid, and the portion of the second liquid is propelled towards the selected region on the substrate by locally coupling energy into a region containing or adjacent to the portion of the second liquid to be propelled towards the selected region on the first substrate. The coupling of energy may comprise locally generating heat or pressure. This approach allows the dividing process to be formed quickly, flexibly and with high resolution. In some embodiments, the local coupling of energy is achieved using a focussed beam of electromagnetic radiation or ultrasound.

In an embodiment, the second liquid is denser than the first liquid.

The method is surprisingly effective using a second liquid that is denser than the first liquid, despite the forces of buoyancy which might be expected to lift the first liquid away from contact with the substrate. Allowing use of a denser second liquid advantageously widens the range of compositions that can be used for the second liquid. Furthermore, the maximum depth of first liquid that can be retained stably in each sub-body without the first liquid spreading laterally over the substrate is increased.

According to an aspect, there is provided a method of operating a microfluidic arrangement, comprising: providing a microfluidic arrangement comprising a continuous body of a first liquid in direct contact with a substrate, and a second liquid in direct contact with the continuous body of first liquid and covering the continuous body of first liquid, the second liquid being immiscible with the first liquid, wherein one or more walls of second liquid are pinned in contact with a selected region of the substrate to define a shape of the continuous body of first liquid, wherein: the one or more walls of second liquid define a plurality of open-ended chambers containing the first liquid; and the method further comprises: providing target material different from the first liquid and the second liquid in each of a plurality of the open-ended chambers; and driving a flow of the first liquid past open ends of the open-ended chambers or through the open-ended chambers.

Thus, a method is provided that allows experiments requiring flow of liquid past or around target material of interest (e.g. biological material) to be constructed and operated flexibly and efficiently.

In an embodiment, the target material is provided in the continuous body of the first liquid before the one or more walls of second liquid are formed. In an embodiment, the target material comprises adherent living cells and at least a portion of the cells are allowed to adhere to the substrate before the one or more walls of second liquid are formed. A reagent (e.g. drug) may be added to the continuous body of the first liquid after at least a portion of the adherent living cells have adhered to the substrate. This methodology allows adhered living cells to be treated en masse after they have been allowed to adhere to a substrate, with the geometry of the open-ended chambers being defined later on. This is not possible using prior art approaches and saves considerable time and system complexity, particularly where it is desired to create large numbers of isolated samples and minimum disruption to the cells. It also ensures that cells in each sample (open-ended chamber) have been exposed to very similar conditions, which is difficult to ensure when test substances (e.g. drugs) are added to individual wells or droplets manually, which may impose significant delays between treatment, and physical environments due to inkjet printing or the drop-seq method, of different samples. The cells can be placed on the surface without the stresses that would be imposed by passing them through a printing nozzle of an inkjet style printing system. Allowing the cells to adhere before forming the one or more walls of second liquid provides a better representation of more classical well plate starting conditions for drug screening than alternative approaches in which cells are brought into miniature volumes before they adhere (e.g. via droplet printing).

According to an alternative aspect, there is provided an apparatus for manufacturing a microfluidic arrangement, comprising: a substrate table configured to hold a substrate on which a continuous body of a first liquid is provided in direct contact with a substrate, and a second liquid is provided in direct contact with the first liquid and covering the first liquid, the second liquid being immiscible with the first liquid; and a pattern forming unit configured to propel a separation fluid, immiscible with the first liquid, through at least the first liquid and into contact with the first substrate over all of a selected region on the surface of the first substrate, thereby displacing first liquid that was initially in contact with the selected region away from the selected region without any solid member contacting the selected region directly and without any solid member contacting the selected region via a globule of liquid held at a tip of the solid member, the selected region being such that one or more walls of second liquid are formed that modify a shape of the continuous body of first liquid.

Thus, an apparatus is provided that is capable of performing methods according to the disclosure.

Embodiments of the invention will now be described, by way of example only, with reference to the accompanying drawings in which corresponding reference symbols indicate corresponding parts, and in which:

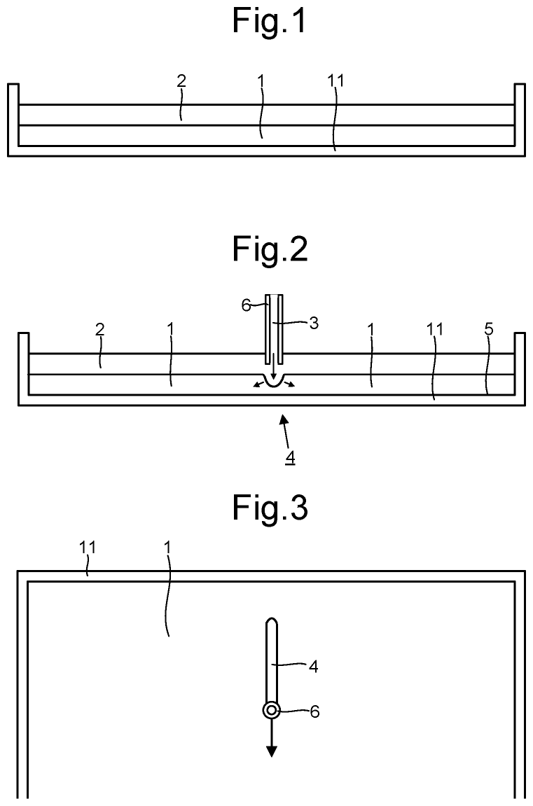

FIG. 1 is a schematic side view of a continuous body of a first liquid on a substrate with a second liquid in direct contact with the first liquid and covering the first liquid;

FIG. 2 is a schematic side view of the arrangement of FIG. 1 during formation of a wall of second liquid by pumping a separation fluid out of a distal tip of an injection member;

FIG. 3 is a schematic top view of the arrangement of FIG. 2;

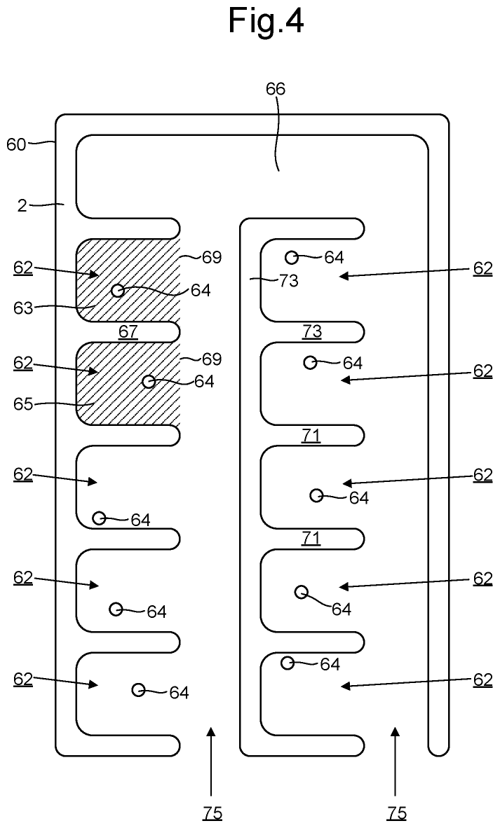

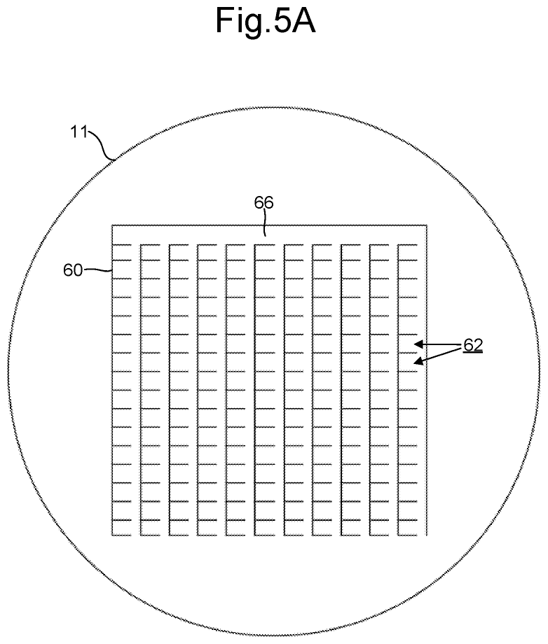

FIG. 4 is a schematic top view showing a microfluidic arrangement comprising a plurality of open-ended chambers formed using the methodology of FIGS. 2 and 3;

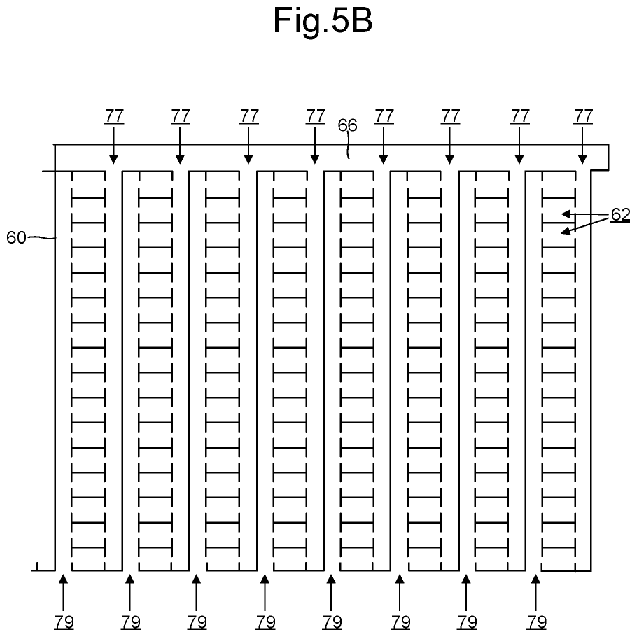

FIG. 5A depicts a network of the type depicted in FIG. 4 with a larger number of chambers;

FIG. 5B depicts an alternative network comprising chambers having two open ends;

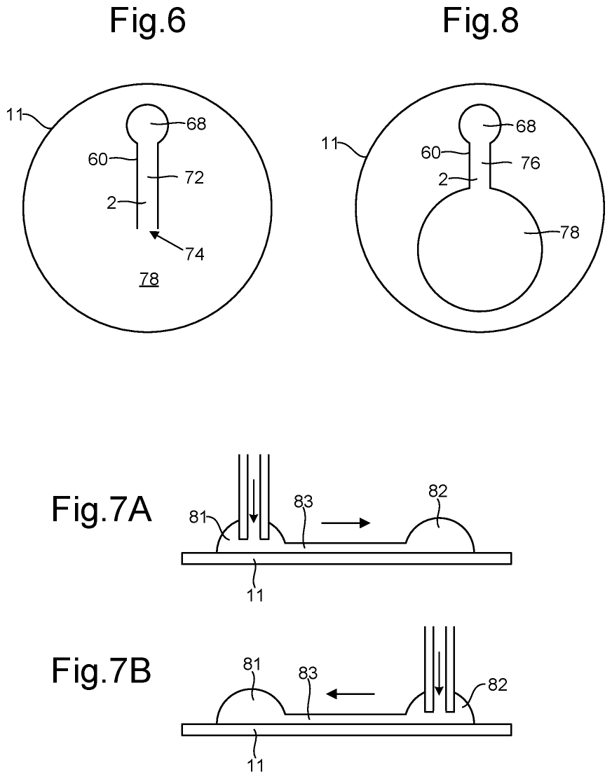

FIG. 6 depicts an open-ended conduit configured to act as a passive check valve;

FIGS. 7A and 7B are schematic top views of a microfluidic arrangement comprising two reservoirs connected together by a flow conduit;

FIG. 8 depicts an alternative configuration for a passive check valve;

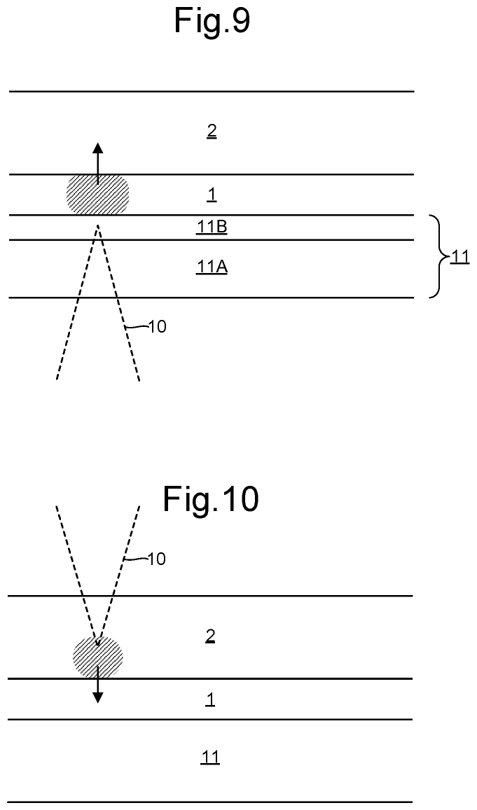

FIG. 9 is a schematic side sectional view showing focusing of a laser beam into an intermediate absorbing layer of a substrate to propel first liquid away from the substrate and thereby allow the second liquid to move into contact with a selected region on the substrate;

FIG. 10 is a schematic side sectional view showing focusing of a laser beam into the second liquid to propel a portion of the second liquid through the first liquid and onto a selected region on the substrate;

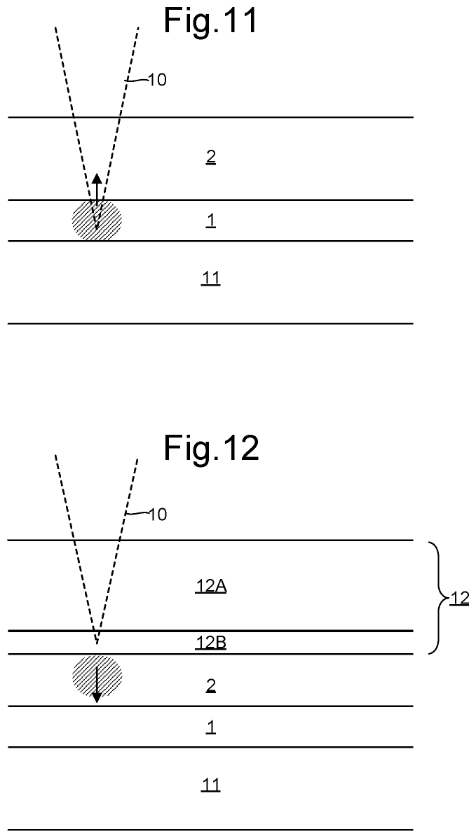

FIG. 11 is a schematic side sectional view showing focusing of a laser beam into the first liquid to propel first liquid away from the substrate and thereby allow the second liquid to move into contact with a selected region on the substrate;

FIG. 12 is a schematic side sectional view showing focusing of a laser beam into an intermediate absorbing layer of a second substrate to propel a portion of the second liquid through the first liquid and onto a selected region on the substrate;

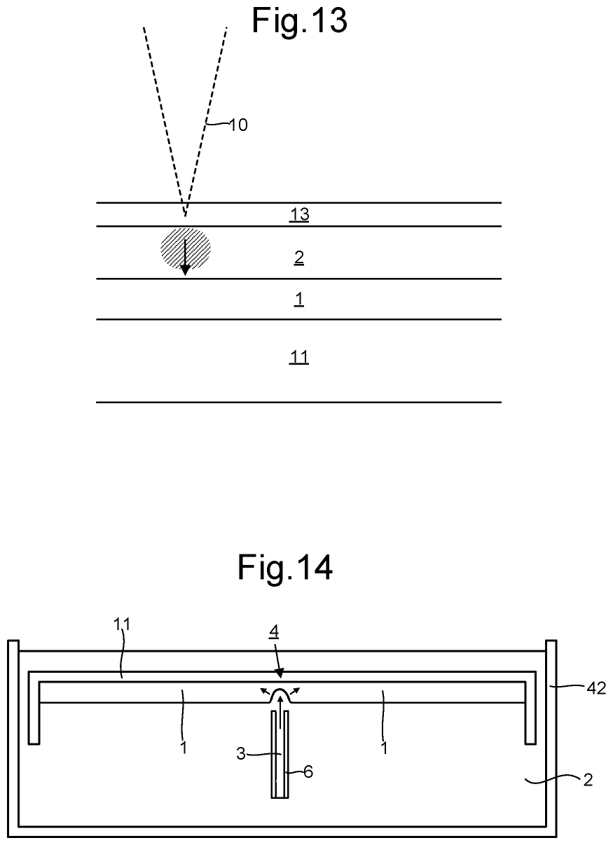

FIG. 13 is a schematic side sectional view showing focusing of a laser beam into a third liquid to propel a portion of the second liquid through the first liquid and onto a selected region on the substrate;

FIG. 14 depicts formation of a wall of second liquid through a continuous body of first liquid while the continuous body is held upside down;

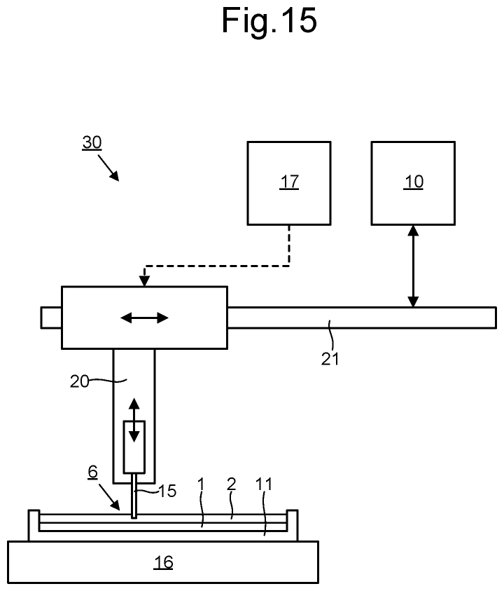

FIG. 15 depicts an apparatus for manufacturing a microfluidic arrangement according to embodiments of the disclosure involving pumping of separation fluid out of a distal tip of an injection member;

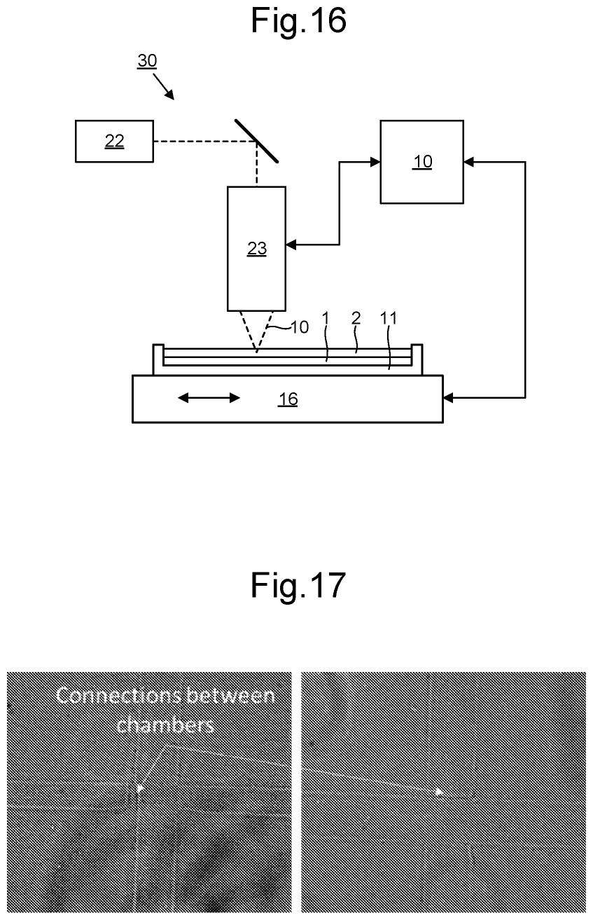

FIG. 16 depicts an apparatus for manufacturing a microfluidic arrangement according to embodiments of the disclosure involving use of a laser beam to propel the separation fluid through the first liquid and into contact with the substrate;

FIG. 17 depicts images of unwanted breaks in walls of liquid formed using an alternative technique; and

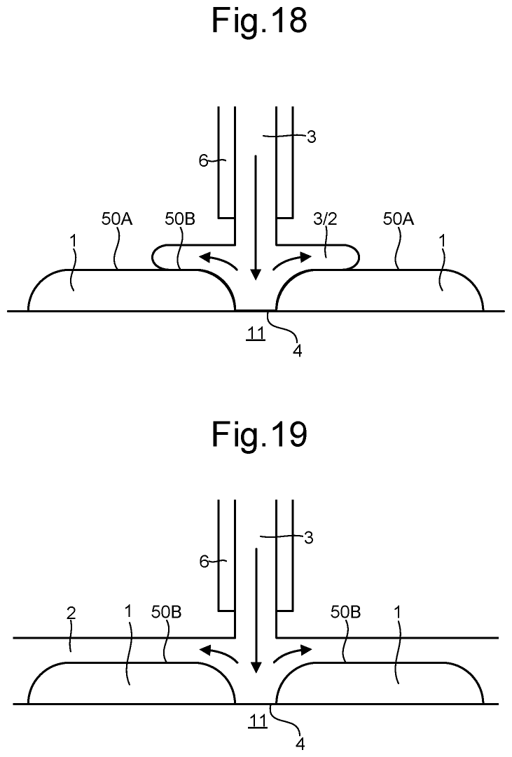

FIGS. 18 and 19 are schematic side sectional views showing steps in a method of manufacturing a microfluidic arrangement in which a separation fluid is propelled initially through a continuous body of first liquid that is not covered by any second liquid; FIG. 18 depicts an initial stage in which the separation fluid is only just starting to cover the first liquid, such that a portion of an upper interface of the first liquid is not yet in contact with any second liquid; FIG. 19 depicts a later stage in which the separation fluid, which may now be referred to as the second liquid, completed covers the first liquid.

The figures are provided for explanatory purposes only and are not depicted to scale in order to allow constituent elements to be visualised clearly. In particular, the width of the receptacle providing the first substrate relative to the depth of the first and second liquids will typically be much larger than depicted in the drawings.

Methods are provided for conveniently and flexibly manufacturing a microfluidic arrangement.

As depicted schematically in FIG. 1, a continuous body of a first liquid 1 is provided. The first liquid 1 is in direct contact with a first substrate 11. In an embodiment the first liquid 1 comprises an aqueous solution but other compositions are possible. A second liquid 2 is provided in direct contact with the first liquid 1. The second liquid 2 is immiscible with the first liquid. In an embodiment, the continuous body of the first liquid 1 is formed on the first substrate 11 before the second liquid 2 is brought into contact with the first liquid 1. In other embodiments, the continuous body of the first liquid 1 is formed after the second liquid 2 is provided (e.g. by injecting the first liquid 1 through the first liquid 2). In embodiments in which the microfluidic arrangement is to be used for testing samples of biological material, the continuous body of the first liquid 1 will normally be formed before the second liquid 2 is provided. The second liquid 2 covers the first liquid 1. The first liquid 1 is thus completely surrounded and in direct contact exclusively with a combination of the second liquid 2 and the first substrate 11 (which, when the substrate 11 is formed from a dish, may include all or a portion of the base of the dish and a portion of a wall of the dish). At this point in the method the first liquid 1 is not in contact with anything other than the second liquid 2 and the first substrate 11. Typically, the first substrate 11 will be unpatterned (neither mechanically nor chemically), at least in the region in contact with the continuous body of the first liquid 1 (typically underneath and/or laterally surrounding). In some embodiments, the first substrate 11 has been plasma treated. In an embodiment, the continuous body of the first liquid 1 is in direct contact on its lower side exclusively with a substantially planar portion of the first substrate 11 and on its upper side exclusively with the second liquid 2. The continuous body of the first liquid 1 may additionally be in direct contact with lateral sides with the first substrate 11 (e.g. where the continuous body of the first liquid 1 extends to lateral side walls of a dish forming the first substrate 11). The continuous body of the first liquid 1 may be provided for example by providing a relatively large volume of the first liquid 1 in a dish and then removing most of the first liquid 1 (e.g. by pouring off or syringing) to leave a thin film of the first liquid 1 in the dish. In a subsequent step, an example implementation of which is depicted in FIG. 2, a separation fluid 3 is propelled through at least the first liquid 1 (and optionally also through a portion of the second liquid 2, as shown in the example of FIG. 2) and into contact with the first substrate 11 over all of a selected region 4 on the surface 5 of the first substrate 11. The selected region 4 consists of a portion of the surface area of the surface 5 of the first substrate 11. The selected region 4 may comprise a path having a finite width. Portions of the selected region 4 may be substantially elongated and interconnected, the selected region thereby forming a network or web-like pattern. The separation fluid 3 is immiscible with the first liquid 1. The separation fluid 3 displaces the first liquid 1 away from the selected region 4 without any solid member contacting the selected region 4 directly (e.g. by dragging a tip of the solid member over the surface of the first substrate 11) and without any solid member contacting the selected region 4 via a globule of liquid held at a tip of the solid member (e.g. by dragging the globule of liquid, held stationary relative to the tip, over the surface of the first substrate 11). The first liquid 1 is initially in contact with (e.g. all of) the selected region 4. The surface area defined by the selected region 4 may therefore represent a portion of the surface area of the first substrate 11 in which the first liquid 1 has been displaced away from contact with the first substrate 11 by the separation fluid 3 that has been propelled through the first liquid 1. In the embodiment of FIG. 2, the separation fluid 3 is propelled (e.g. by pumping) onto the selected region 4 from a lumen in a distal tip 6 of an injection member while the distal tip 6 is moved relative to (e.g. scanned over) the first substrate 11. No contact is therefore made in this embodiment between the distal tip 6 and the selected region 4 during movement of the distal tip 6 over at least a portion of the selected region 4. No contact is made by the selected region 4 with any other solid member, either directly or via a globule of liquid that is stationary relative to the solid member, for at least a portion of the selected region 4. The momentum of the separation fluid 3 is sufficient to force the first liquid 1 to be displaced away from the selected region 4. In an embodiment, the separation fluid 3 is pumped continuously out of the distal tip for at least a portion of the selected region. In the embodiment shown in FIG. 2, the separation fluid 3 is pumped out of the distal tip 6 in a direction that is substantially perpendicularly to the selected region 4 at the location of the distal tip 6. In other embodiments, the distal tip 6 may be tilted so as to pump the separation fluid 3 towards the selected region 4 at an oblique angle relative to the selected region 4.

In an embodiment, the selected region 4 is such that one or more walls of second liquid 2 are formed that modify a shape of the continuous body of first liquid 1. The second liquid 2 moves into contact with the selected region 4 and remains stably in contact with the selected region 4. A pinning line (associated with interfacial forces) stably holds the footprints of one or more walls of second liquid 2 in place. The footprints of walls are pinned in a static configuration by interfacial forces. The pinning is such that each of the walls of second liquid 2 has a wall footprint representing an area of contact between the second liquid 2 of the wall and the first substrate 1 that remains constant even when liquid is added to or removed from the microfluidic arrangement (the liquid walls morph above the unchanging footprint to accommodate the addition or removal). The first liquid 1 and the second liquid 2 remain in liquid form. Various combinations of materials for the first liquid 1, second liquid 2 and first substrate 11 enable this stable pinning to occur.

The one or more walls of second liquid 2 define features of the microfluidic arrangement. In an embodiment, the features comprise one or more closed features, thereby defining sub-bodies of the first liquid 1 formed by dividing the continuous body of first liquid 1 into a plurality of sub-bodies of the first liquid 1 via the one or more walls of second liquid 2. Each sub-body is separated from each other sub-body by the second liquid 2. Such a plurality of sub-bodies may comprise a single useful sub-body and a remainder of the continuous body of the first liquid 1 (which may be considered as another sub-body) or may comprise plural useful sub-bodies (e.g. plural reservoirs for receiving reagents etc.), optionally together with any remainder of the continuous body of the first liquid 1.

In an embodiment, the features comprise one or more open features. The open features may include, for example, open-ended flow conduits or open-ended chambers. The flow conduits may comprise portions of the first liquid 1 that are constrained by the one or more walls of second liquid to adopt an elongate shape (e.g. surrounded laterally and from above by the second liquid and from below by the first substrate 11). The continuous body of first liquid 1 may thus remain a single continuous body of first liquid 1 after the modification of the shape of the continuous body of first liquid 1 by the one or more walls of second liquid 2. The continuous body of first liquid 1 is continuous in that every point in the continuous body of first liquid is connected to every other point in the continuous body of first liquid 1 along an uninterrupted path going exclusively through the first liquid 1. The continuous body of first liquid 1 is not divided into isolated sub-bodies in embodiments of this type.

In an embodiment, the one or more walls of second liquid 2 define a plurality of open-ended chambers 62. Examples of an arrangement of this type are depicted in FIGS. 4, 5A and 5B. FIG. 4 depicts a relatively small example with only 10 open-ended chambers 62. FIG. 5A depicts an example with a larger number of open-ended chambers 62. FIG. 5B depicts a variation in which at least a subset of the open-ended chambers 62 have two open ends and the one or more walls of second liquid 2 are further configured to direct a flow of the first liquid 1 through each of the open-ended chambers 62 having two open ends. Practical embodiments may contain even more chambers than the examples shown, for example 100s or 1000s of chambers. Each open-ended chamber 62 contains the first liquid 1 and is separated from each other open-ended chamber 62 of at least a first plurality of the open-ended chambers 62 by the one or more walls of second liquid 2. The separation is to the extent that there is no uninterrupted straight line path through the first liquid 1 from the inside of any one of the open-ended chambers 62 of at least the first plurality of open-ended chambers 62 to the inside of any other one of the open-ended chambers 62 of at least the first plurality of open-ended chambers 62. Thus, for example, none of the first liquid 1 in the hatched region 63 of an open-ended chamber 62 in FIG. 4 can flow in a straight line into the hatched region 65 of the nearest other open-ended chamber 62. The straight line flow is prevented by the portion 67 of the wall of second liquid 2 separating the two open-ended chambers 62. Each chamber 62 is, however, open-ended in the sense that the chamber 62 comprises at least one open-end 69 via which first liquid 1 can enter or leave the open-ended chamber 62 without being prevented from doing so by a wall of the second liquid 2, and hence diffusion through the first liquid 1 is possible between different chambers 62.

In an embodiment, the one or more walls of second liquid 2 define a first plurality of the open-ended chambers 62 and a second plurality of the open-ended chambers 62. The first plurality of open-ended chambers 62 does not include any of the open-ended chambers 62 of the second plurality of open-ended chambers 62. The first plurality of open-ended chambers 62 are separated from each other in the sense described above with reference to FIG. 4 (i.e. such that there is no uninterrupted straight line path through the first liquid 1 from the inside of any one of those open-ended chambers 62 to the inside of any other one of those open-ended chambers 62) and the second plurality of open-ended chambers 62 are separated from each other in the sense described above with reference to FIG. 4 (i.e. such that there is no uninterrupted straight line path through the first liquid 1 from the inside of any one of those open-ended chambers 62 to the inside of any other one of those open-ended chambers 62). The first plurality of open-ended chambers 62 are not, however, necessarily separated from all of the second plurality of open-ended chambers 62 in the same sense. This may be the case, for example, where the one or more walls of second liquid 2 define a flow conduit that allows a flow of the first liquid 1 to be driven past the open ends of both of the first plurality of open-ended chambers 62 and the second plurality of open-ended chambers 62 and open ends of different chambers 62 face each other across the flow conduit.

In an embodiment, as is the case in the examples of FIGS. 4, 5A and 5B, an outline of the wall footprint 60 of at least one of the walls comprises at least one straight line segment (see the portion 67 of the wall in FIG. 4 for example). Straight line segments can be formed efficiently by an appropriate scanning action of a distal tip. Straight line segments allow higher space filling efficiency in comparison with geometries defined, for example, by circular or elliptical bodies of liquid. In an embodiment, the wall footprint 60 comprises multiple linear portions that are parallel to each other, such as the portions labelled 71 in FIG. 4. In an embodiment, the wall footprint 60 comprises linear portions that intersect each other at right angles (perpendicularly), such as the portions labelled 73 in FIG. 4. An outline of the wall footprint 60 in this case will comprise at least two straight line segments that are perpendicular to each other. The straight line segments may form portions of square, rectangular or other tessellating shapes for example.

The microfluidic arrangement of FIG. 4 is an example of a microfluidic arrangement that can be used in a method of operating a microfluidic arrangement according to an embodiment. The microfluidic arrangement may be manufactured in accordance with any embodiment of the present disclosure. The microfluidic arrangement may thus comprise a continuous body of a first liquid 1 in direct contact with a substrate 11, and with a second liquid 2 in direct contact with the continuous body of first liquid 1 and covering the continuous body of first liquid 1. One or more walls of the second liquid 2 may be pinned in contact with a selected region 4 of the substrate 11 to define a shape of the continuous body of first liquid 1. The one or more walls of second liquid may define a plurality of open-ended chambers. In this embodiment, biological material (such as cells, DNA, proteins, etc.) to be investigated may be provided in each of a plurality of the open-ended chambers 62. In an embodiment, the biological material comprises adherent living cells. In an embodiment, one or more living cells 64 are provided in each of a plurality of the open-ended chambers 62. In the example shown, one cell 64 is provided in each of the available open-ended chambers 62. In other embodiments, one cell 64 is only provided in a subset of the available open-ended chambers 62 (i.e. in fewer than all of them). In other embodiments, more than one cell is provided in one or more of the open-ended chambers 62. In an embodiment, a flow of the first liquid is driven past the open ends 69 of the open-ended chambers 62 containing the deposited cells 64. The one or more walls of second liquid 2 define one or more flow conduits 75 allowing a flow of the first liquid 1 to be driven past the open ends 69 of the open-ended chambers 62. The flow of the first liquid 1 may be driven in various ways. For example, liquid could be pumped into the input region 66 in FIGS. 4 and 5A, which would lead to first liquid 1 flowing generally downwards along the flow conduits 75. In the embodiment of FIG. 5B having open-ended chambers 62 with two open ends, the flow of the first liquid 1 may be driven by pumping liquid into the input region 66, which would lead to the first liquid 1 flowing downwards along flow conduits 77, laterally through the open-ended chambers 62 and downwards along flow conduits 79. Various experiments using such a controlled flow of liquid past living cells are desirable, including for example perfusion experiments. For example, human cells are often cultured for days when growth requires addition of fresh medium and removal of waste material. If cells 64 are contained in open-ended chambers 62, pumping fresh medium into input region 66 would induce flow down through flow conduits 75, and diffusional exchange would refresh open-ended chambers 62 and remove waste from them.

In an embodiment, pumping into input region 66 is performed using a hydrostatic head, which is cheap to implement in comparison with an active pump. In an embodiment, the flow of the first liquid 1 is driven constantly or pseudo-constantly (e.g. in a pulsed manner with small time intervals between consecutive pulses) to maintain the volumes of the open-ended chambers 62 within a desired range and/or to provide sufficient fresh medium and/or waste removal. The flow causes an increase in pressure in the first liquid 1 which makes the corresponding portions of the microfluidic arrangement (e.g. flow conduits 77 and chambers 62) larger (taller). The flow may also provide a continuous replacement of nutrients. Some cells typically do not need flow per se, and can be maintained in static chambers (e.g. in a traditional well plate). However, the volume of such static chambers limits the time that the cells can be maintained without replenishing nutrients. Smaller chambers will need to be replenished sooner than larger chambers. Providing a constant or pseudo-constant flow past or through chambers containing cells provides behaviour analogous to an infinitely large chamber, in that nutrients can be continuously supplied without needing separate nutrient replenishing actions. Other cells are best cultured in a flowing (and sometimes pulsatile) environment, for example the endothelial cells of arteries and veins. Providing cells close to or within flows of liquid containing nutrients also more closely resembles the environment within the body than providing cells in isolated liquid chambers (e.g. as in a traditional well plate).

In an embodiment, the substrate 11 is tilted so a number of cells 64 freshly-deposited in one of the chambers 62 can become concentrated by gravity as they settle into one corner at the closed end of chamber 62. This is attractive: (a) e.g., to reduce the likelihood that non-adherent cells are inadvertently removed with waste when a tube is inserted centrally in a chamber 62 and medium withdrawn; and (b) e.g., because one wants to aggregate a suspension of single cells of the same type to create a spheroid or embryoid body—a three-dimensional aggregate of cells in which cells in different parts of the aggregate become different from each other in much the same way that different parts of an embryo develop into heart and brain cells. Creation of spheroids or embryoid bodies is a step often found in the pathway from an induced pluripotent cell to a differentiated cell like a neuron or muscle cell, and apparatus to facilitate this step have been developed (e.g. the ‘AggreWell™’ of StemCell Technologies; https://www.stemcell.com/products/brands/aggrewell-3d-culture.html).

In an embodiment, fresh medium is pumped into input region 66, flows down through flow conduits 75 and out of the system to a region where the medium rises due to buoyancy and detaches from the microfluidic arrangement to form a layer above the second liquid 2, thereby allowing the microfluidic arrangement to self empty.

More general benefits of arrangements comprising the open-ended chambers 62 in comparison with prior art alternatives include: the ability to use the same materials for the substrate 11 that have been used for many years in similar biological experiments, thereby avoiding unexpected interactions with biological material; the intrinsic removal of gases; and open access to all parts of the microfluidic arrangement (without having to deal with solid walls for example).

In an embodiment, the biological material is provided in the continuous body of the first liquid 1 before the one or more walls of second liquid 2 are formed. This approach allows multiple chambers 62 containing biological material to be formed without the biological material needing to be added individually to each chamber 62, which would be very time consuming, particularly where large numbers of chambers 62 are used and/or where the chambers 62 are very small. This approach could be used with non-adherent living cells. This approach is particularly advantageous where the biological material comprises adhered living cells because it allows adhered living cells to be treated en masse after they have been allowed to adhere to a substrate, and divided into the chambers 62 later on. This is not possible using prior art approaches and saves considerable time and system complexity, particularly where it is desired to create large numbers of samples.

FIG. 6 is a top view of a microfluidic arrangement in which the one or more walls of second liquid 2 define an open-ended flow conduit 72. Other microfluidic elements can be connected to the open-ended flow conduit 72 at an end of the open-ended flow conduit 72 opposite to the open end 74. In the example of FIG. 6, an input reservoir 68 is provided. The open end 74 of the open-ended flow conduit 72 opens into a macroscopic sink volume 78. The input reservoir 68 may comprise a generally hemispherical body of first liquid 1. The open-ended flow conduit 72 may comprise a generally elongate body of first liquid 1 with a generally semi-circular cross-section. The open-ended flow conduit 72 is configured so that in use flow can be driven forwards through the open-ended flow conduit 72 by adding a volume of liquid to the microfluidic arrangement upstream of the open end 74 but the addition of the same volume of liquid into the macroscopic sink volume 78 will not drive any significant flow along the open-ended flow conduit 72 in the opposite direction. The open end 74 of the open-ended flow conduit 72 thus acts in a similar way to a check valve with respect to addition of liquid to regions upstream and downstream of the open end 74, with no moving parts or power input being needed to effect the functionality. The functionality relies on the macroscopic sink volume 68 having a very much larger volume than any reservoir directly connected upstream of the open-ended flow conduit 72. The relatively small volumes present in the microfluidic arrangement upstream from the open end 74 of the open-ended flow conduit 72 effectively define a “micro-world” in comparison with the “macro-world” defined by the much larger volume associated with the macroscopic sink reservoir 78 downstream from the open end 74 of the open-ended flow conduit 72.

Microvalves are widely required in microfluidics. This is discussed for example in “Au, A. K., Lai, H., Utela, B. R., and Folch, A. (2011). Microvalves and micropumps for BioMEMS. Micromachines 2, 179-220” and in “Oh, K. W., and Ahn, C. H. (2006). A review of microvalves. J. Micromech. Microeng. 16, R13-39”. Check valves can be characterized in three ways: (i) active check valves actuated by external forces, (ii) passive check valves (e.g., ‘Domino valves’ actuated by fluid motion), and (iii) fixed-geometry check valves that have no moving parts or deformable structures and so do not require external power (e.g., a ‘Tesla valve’ or ‘valvular conduit’ that allows easy passage of forward flow but discourages reverse flow). The latter two alternatives are sometimes referred to as fluid diodes. Compared with such arrangements and others, the use of open-ended conduits 72 to implement similar functionality (in the manner described above) provides improved simplicity (e.g. no moving parts and no energy requirements for operation), greater ease and/or lower cost of manufacture and operation, and/or high effectiveness (back flow can be stopped completely or to a very high degree, which is not achieved in Tesla valves for example).

FIGS. 7A and 7B depict a simple circuit comprising a first reservoir 81 and a second reservoir 82 connected together by a flow conduit 83. All three bodies may be formed by walls of second liquid 2 as described above. In a circuit of this type it is possible to drive a flow of liquid in both directions. In other words, if (as depicted in FIG. 7A) one inserts a tube connected to syringe pump into the first reservoir 81 (acting as a source reservoir) and then drives flow to the second reservoir 82 (acting as a sink reservoir), flow will continue until pressures equalize in the two reservoirs 81 and 82 (or the circuit ruptures). The same applies if flow is driven by a hydrostatic head or a difference in Laplace pressure. If (as depicted in FIG. 7B) one now inserts the tube into the second reservoir 82 (which was previously the sink reservoir), one can drive flow the other way (as there are no valves in the system). Again flow will continue until pressures equalize or the circuit ruptures.

Comparing the microfluidic arrangement of FIG. 6 with the arrangement of FIGS. 7A and 7B, the input reservoir 68 corresponds most closely to the reservoir 81 in FIG. 7A and to the reservoir 82 in FIG. 7B. The open-ended flow conduit 72 corresponds most closely to the flow conduit 83. The macroscopic sink reservoir 78 corresponds most closely to the reservoir 82 in FIG. 7A and to the reservoir 81 in FIG. 7B. The gap between the two walls at the open end 74 of the open-ended flow conduit 72 lies at the interface between the micro- and macro-worlds. If liquid is pumped into the input reservoir 68 there will be a flow of liquid through the open-ended conduit 72 and out of the open end 74 to the volume outside, where the liquid involved in this flow can accumulate either as a relatively flat drop in the volume, or at the edge of the volume where it may form a meniscus against the side of the container (e.g. dish) providing the substrate 11. This flat drop has very low curvature. If the pumping is stopped, Laplace pressure continues to drive forward flow to the macroscopic sink reservoir 78. This will continue for some time as flow through the micro-world part is slow. To allow self-emptying, one could draw a hydrophilic line up the edge of the container/dish (or fit a tube at the edge) to allow buoyancy to drive the liquid involved in the flow above the second liquid 2.

If one now inserts the tube into the macroscopic sink reservoir 78 and starts pumping, initially flow will not be back through the open end 74 of the open-ended conduit 72 into the input reservoir 68 (because the open-ended conduit 72 and/or input reservoir 68 has/have a relatively large positive curvature and the macroscopic sink reservoir 78 has extremely small and/or zero and/or negative curvature). Instead, the extra liquid is accommodated in the macroscopic sink reservoir 78. The level will rise to create a hydrostatic head, but this happens only extremely slowly and does not create any significant back flow in timescales relevant to the experiments being performed. The arrangement is more effective and simpler than, for example, a Tesla valve (which does not completely stop backflow from the beginning).

The particular compositions of the first liquid 1, second liquid 2, the separation fluid and first substrate 11 are not particularly limited. However, it is desirable that the first liquid 1 and the second liquid 2 can wet the first substrate 11 sufficiently for the method to operate efficiently. Furthermore, it is desirable that no phase change occurs during the manufacturing of the microfluidic arrangement. For example, the separation fluid, first liquid 1 and second liquid 2 may all be liquid before the microfluidic arrangement is formed and remain liquid during the manufacturing process and for a prolonged period after the microfluidic arrangement is formed and during normal use of the microfluidic arrangement. In an embodiment, the first liquid 1, second liquid 2 and first substrate 11 are selected such that an equilibrium contact angle of a droplet of the first liquid 1 on the first substrate 11 in air and an equilibrium contact angle of a droplet of the second liquid 2 on the first substrate 11 in air would both be less than 90 degrees. In an embodiment, the first liquid 1 comprises an aqueous solution. In this case the first substrate 11 could be described as hydrophilic. In an embodiment, the second liquid 2 comprises a fluorocarbon such as FC40 (described in further detail below). In this case the first substrate 11 could be described as fluorophilic. In the case where the first liquid 1 is an aqueous solution and the second liquid 2 is a fluorocarbon, the first substrate 11 could therefore be described as being both hydrophilic and fluorophilic.

The separation fluid 3 may comprise one or more of the following: a gas, a liquid, a liquid having the same composition as the second liquid 2, a portion of the second liquid 2 provided before the propulsion of the separation fluid 3 through the first liquid 1.

In some embodiments, as mentioned above, the separation fluid 3 is propelled onto the selected region 4 on the first substrate 11 from a lumen (e.g. by continuously pumping the separation fluid 3 out of the lumen, optionally at a substantially constant rate) in a distal tip 6 of an injection member while the distal tip 6 is moved relative to (e.g. scanned over or under along a path corresponding to the selected region 4) the first substrate 11 (with some first liquid 1 and, optionally, second liquid 2, between the distal tip 6 and the first substrate 11). In some embodiments of this type, the distal tip 6 is moved through both of the second liquid 2 and the first liquid 1 while propelling the separation fluid 3 onto the selected region 4 on the first substrate 11, for at least a portion of the selected region 4. The distal tip 6 is thus held relatively close to the first substrate 11. In such embodiments, the movement of the distal tip 6 and the flow of the separation fluid 3 towards the first substrate 11 both act to displace the first liquid 1 away from the first substrate 11, allowing the second liquid 2 to move into the volume previously occupied by the first liquid 1. In an embodiment, this process is facilitated by arranging for at least a portion of the distal tip 6 to be more easily wetted by the second liquid 2 than by the first liquid 1. In this way, it is energetically more favourable for the second liquid 2 to flow into the region behind the moving distal tip 6 and thereby displace the first liquid 1 efficiently. Preferably the first substrate 11 is also configured so that it is more easily wetted by the second liquid 2 than by the first liquid 1, thereby energetically favouring contact between the second liquid 2 and the first substrate 11 along the selected region 4. This helps to maintain a stable arrangement in which the walls of second liquid 2 are stably pinned in place. In other embodiments, an example of which is shown in FIG. 2, the distal tip 6 is moved through the second liquid 2 but not the first liquid 1 while propelling the separation fluid 3 onto the selected region 4 on the first substrate 11, for at least a portion of the selected region 4. The distal tip 6 is thus held further away from the first substrate 11. This approach helps to avoid detachment of droplets of the first liquid 1 from the first substrate 11 caused by the pumping of the separation fluid 3 against the first substrate 11.

FIGS. 2-3 illustrate an example embodiment in which a distal tip 6 moves through the second liquid 2 but not the first liquid 1 in a horizontal direction, parallel (in this example) to a plane of the first substrate 11 that is in contact with the first liquid 1. Separation fluid 3 is pumped from the distal tip 6. The vertical arrow exiting the distal tip 6 in FIG. 2 schematically represents an example pumped flow of the separation fluid 3 (note that the pumped flow does not need to be vertical; oblique angles of incidence may also be used, with an angle even being be used, optionally, to control the width of walls of second liquid 2 that are formed). Arrows within the first liquid 1 in FIG. 2 schematically represent movement of the first liquid 1 away from the region above a portion of the selected region 4, which will eventually allow the second liquid 2 to contact the first substrate 11 along the selected region 4. In FIG. 2, the movement of the distal tip 6 is into the page. In FIG. 3, the movement is downwards. In an embodiment, the distal tip 6 is maintained at a constant distance from the first substrate 11 while the distal tip 6 is being moved through the second liquid 2. The process of FIGS. 2 and 3 could be continued to an end of the continuous body of first liquid 1 to divide the continuous body of the first liquid 1 of FIG. 1 into two sub-bodies and/or repeated and/or performed in parallel to create a desired number and size of individual sub-bodies. The pumping of the separation fluid 3 is optionally stopped and started between movement of the distal tip 6 over different portions of the selected region, or the pumping may continue as the distal tip moves from the end of one portion of the selected region to the start of the next portion of the selected region. The steps of FIGS. 2 and 3 can be repeated to form multiple parallel lines of a selected region 4 (with the pumping of the separation fluid 3 being optionally stopped and started between formation of each of the parallel lines, or the pumping may continue while the distal tip moves from the end of one parallel line to the start of the next parallel line). By repeating the process in the orthogonal direction multiple square sub-bodies could be provided. In practice, many 100s or 1000s of sub-bodies could be provided in this manner. The inventors have demonstrated for example that the approach can be used routinely to obtain a square array of sub-bodies having a pitch of less than 100 microns. This is considerably smaller than would be possible using standard microwell plate manufacturing techniques.

In an embodiment, the selected region 4 is such that, for each of one or more sub-bodies defined by the one or more walls of second liquid 2, a sub-body footprint represents an area of contact between the sub-body and the first substrate 11 and all of a boundary of the sub-body footprint is in contact with a closed loop of the selected region 4 surrounding the sub-body footprint. The closed loop of the selected region 4 is defined as any region that represents a portion of the surface area of the first substrate 11 that forms part of the selected region 4, that forms a closed loop, and that is in contact with the boundary of sub-body along all of the boundary of the sub-body. The first liquid 1, second liquid 2 and first substrate 11 are configured (e.g. by selecting their compositions) such that each boundary of a sub-body footprint that is all in contact with a closed loop of the selected region 4 is pinned in a static configuration by interfacial forces, with the first liquid 1 and second liquid 2 remaining in liquid form. Thus, interfacial forces, which may also be referred to as surface tension, establish pinning lines that cause the sub-body footprints to maintain their shape. The stability of the sub-bodies formed in this way is such that liquid can be added to or removed from each sub-body, within limits defined by the advancing and receding contact angles along the boundary, without changing the sub-body footprint. In some embodiments the boundary of the sub-body footprint that is all in contact with the closed loop of the selected region 4 is made continuously (i.e. in a single process without interruption) and in other embodiments multiple separate steps are used.

In some embodiments, the separation fluid 3 comprises a portion of the second liquid 2 and the portion of the second liquid 2 is propelled towards the selected region 4 by locally coupling energy into a region containing or adjacent to the portion of the second liquid 2 to be propelled towards the selected region 4 on the first substrate 11. The energy coupling may comprise locally generating heat or pressure. The energy may cause expansion, deformation, break-down, ablation or cavitation of material that results in a pressure wave being transmitted towards the portion of the second liquid 2 to be propelled. In some embodiments, the coupling of energy is implemented using a focussed beam of a wave such as electromagnetic radiation or ultrasound. The coupling of energy may occur at or near a focus of the beam.

In an embodiment, a focus of the beam is scanned along a scanning path based on (e.g. following) the geometry of the selected region 4. When viewed perpendicularly to a surface of the first substrate 11 on which the selected region 4 is formed, the scanning path may overlap with at least a portion of the selected region 4 and/or run parallel to at least a portion of the selected region. All or a majority of the scanning path may be below, above or at the same level as the selected region 4 (and, therefore, the surface of the first substrate 11).

In some embodiments, energy from the beam absorbed in the first substrate 11 causes the first liquid 1 to be locally forced away from the first substrate 11 along the selected region 4, the second liquid 2 moving into contact with the first substrate 11 where the first liquid 1 has been forced away (i.e. along the selected region 4). The absorption of the beam in the first substrate 11 may cause local deformation or ablation of the first substrate 11, the localized deformation or ablation transmitting a corresponding localized thrust to first liquid 1 initially in contact with a respective portion of the selected region on the first substrate 11. Using a laser to apply localized thrust to liquids is described in the context of forward printing (i.e. where matter is transferred onto an initially unpatterned substrate to provide a pattern) in, for example, A. Piqué et al. “Direct writing of electronic and sensor materials using a laser transfer technique,” J. Mater. Res. 15(9), 1872-1875 (2000). Methods using this approach have been referred to as laser-induced forward transfer (LIFT) methods. The inventors have recognised that these techniques could be adapted to form one or more walls of second liquid 2 through a continuous body of a first liquid 1 as described herein.

An example of such a configuration is depicted schematically in FIG. 9. In this example, the first substrate 11 comprises a first base layer 11A and a first intermediate absorbing layer 11B between the first base layer 11A and the first liquid 1. A beam absorbance per unit thickness of the first intermediate absorbing layer 11B is higher than a beam absorbance per unit thickness of the first base layer 11A. Energy from the beam absorbed in the first intermediate absorbing layer 11B causes the first liquid 1 to be locally forced away from the first substrate 11 along the selected region 4. A portion of the first liquid 1 to be locally forced away is schematically indicated by hatching in FIG. 9. The second liquid 2 moves into contact with the first substrate 11 where the first liquid 1 has been forced away. The provision of an intermediate absorbing layer 11B that is more absorbing than the base layer 11A provides greater flexibility for choosing a composition of the first substrate 11. For example, the first substrate 11 can be formed predominantly from a material that is relatively transparent to the beam but optimized for other properties, while the first intermediate absorbing layer 11B, which can be provided as a thin film, can be configured specifically to provide a level of absorption and/or other properties that promote efficient localized forcing of the first liquid 1 away from the first substrate 11. In an embodiment, as depicted in FIG. 9, the beam is focused within the first substrate 11 and optionally, where provided, within the first intermediate absorbing layer 11B, to maximise absorption in the first substrate 11 and/or allow the overall beam intensity to be kept as low as possible while still imparting sufficient localized thrust to the first liquid 1. Minimizing the overall beam intensity may be particularly desirable when the first liquid 1 contains material, such as biological material (e.g. cells), that may be adversely affected by the beam. In the example of FIG. 9, the beam 10 is applied from a side of the first substrate 11 opposite to the first liquid 1 and second liquid 2 (i.e. from below in the orientation of FIG. 9). In other embodiments, the beam 10 may be applied from the other side of the first substrate 11, thereby traversing the second liquid 2 before interacting with the first substrate 11.

FIG. 10 depicts an example of an alternative embodiment in which a focus of the beam 10 is positioned within the second liquid 2 while the portion of the second liquid 2 is propelled towards the selected region 4 on the first substrate 11. In some embodiments of this type, the beam causes cavitation in a localized region of the second liquid 2. The cavitation occurs when the absorption in the second liquid 2 is high enough to overcome the optical breakdown threshold of the second liquid 2, which results in generation of a plasma that induces formation of a cavitation bubble. The beam should ideally be tightly focussed with very short laser pulses (e.g. sub-picosecond laser pulses). The cavitation bubble expands and applies a thrust to second liquid 2 in neighbouring regions. If the focus of the beam is positioned adjacent to a portion of the selected region 4, the thrust applied to the neighbouring regions of the second liquid 2 can propel a portion of the second liquid 2 (depicted schematically by hatching in FIG. 10) through the first liquid 1 and into contact with the selected region 4. A diode pumped Yb:KYW femtosecond laser (1027 nm wavelength, 450 fs pulse duration, 1 kHz maximum repetition rate) having a beam waist of around 1.2 microns could be used, for example, as per M. Duocastella et al., “Film-free laser forward printing of transparent and weakly absorbing liquids” OPTICS EXPRESS 11 October 2010/Vol. 18, No. 21 pages 21815-21825, which describes propulsion of droplets via laser induced cavitation within a liquid for the purpose of forward printing droplets from a body of liquid onto a substrate facing the body of liquid. It will be understood that various deviations from the exact laser specifications above could be applied without departing from the underlying principle of operation.

FIG. 11 depicts a variation of the approach depicted in FIG. 10 in which the beam 10 propels the second liquid 2 by causing cavitation in the first liquid 1, the cavitation causing the first liquid 1 to be locally forced away from the first substrate 11, the second liquid 2 moving into contact with the first substrate 11 where the first liquid 1 has been forced away. This may be achieved for example by focussing the beam within the first liquid 1. The portion of the first liquid 1 propelled away from the first substrate 11 by cavitation is depicted schematically by hatching in FIG. 11.

FIG. 12 depicts an example of an alternative embodiment in which a second substrate 12 is provided. The second substrate 12 faces at least a portion of the first substrate 11 and is in contact with liquid. There is a continuous liquid path between the second substrate 12 and the first substrate 11. In the example shown, the second substrate 12 is in contact with the second liquid 2. In this embodiment, energy from the beam 10 is absorbed in either or both of the second substrate 12 and liquid adjacent to the second substrate 12 and causes the second liquid 2 to be locally forced away from the second substrate 12, thereby providing the propulsion of the second liquid 2 towards the selected region 4 on the first substrate 11. In the example shown, the second substrate 12 comprises a second base layer 12A and a second intermediate absorbing layer 12B between the second base layer 12A and the second liquid 2. A beam absorbance per unit thickness of the second intermediate absorbing layer 12B is higher than that of the second base layer 12A. Energy from the beam absorbed in the second intermediate absorbing layer 12B causes the second liquid 2 to be locally forced away from the second substrate 12, thereby providing the propulsion of the second liquid 2 towards the selected region on the first substrate 11. In an embodiment, as depicted in FIG. 12, the beam 10 is focused within the second substrate 12 and optionally, where provided, within the second intermediate absorbing layer 12B, to maximise absorption in the second substrate 12 and/or allow the overall beam intensity to be kept as low as possible while still imparting sufficient localized thrust to the second liquid 2.

In an embodiment, the second substrate 12 floats on liquid (e.g. the second liquid 2) in contact with the second substrate 12. This approach allows the second substrate 12 to be levelled easily and reliably, thereby facilitating accurate alignment of a focus position within the second substrate 12 (e.g. within a second intermediate absorbing layer 12B).

FIG. 13 depicts a variation on the embodiment discussed above with reference to FIG. 12 in which a layer of third liquid 13 is provided above the second liquid 2. A beam absorbance per unit thickness of the third liquid 13 is higher than a beam absorbance per unit thickness of the second liquid 2. Energy from the beam 10 absorbed in the third liquid 13 causes the second liquid 2 to be locally propelled towards the selected region 4 on the first substrate 11. Using a third liquid 13 having higher absorbance than the second liquid 2 provides greater flexibility for choosing the composition of the second liquid 2. The second liquid 2 can be optimized to provide stable formation of the walls of second liquid 2, for example, without being restricted by the need to provide sufficient absorbance to allow the beam to cause cavitation in the second liquid 2 for propelling the second liquid 2 through the first liquid 1. The third liquid 13 can be optimized for absorbing the beam and initiating the formation of a cavitation bubble for locally propelling the second liquid 2 towards the first substrate 11.

In an embodiment, the second liquid 2 is denser than the first liquid 1. The inventors have found that despite the buoyancy forces imposed on the first liquid 1 by the denser second liquid 2 above the first liquid 1, the first liquid 1 surprisingly remains stably in contact with the first substrate 11 due to surface tension effects (interfacial energies) between the first liquid 1 and the first substrate 11. Allowing use of a denser second liquid 2 is advantageous because it widens the range of compositions that are possible for the second liquid 2. For example, in a case where the first liquid 1 is an aqueous solution, a fluorocarbon such as FC40 can be used, which provides a high enough permeability to allow exchange of vital gases between cells in the microfluidic arrangement and the surrounding atmosphere through the layer of the second liquid 2. FC40 is a transparent fully fluorinated liquid of density 1.8555 g/ml that is widely used in droplet-based microfluidics. Using a second liquid 2 that is denser than the first liquid 1 is also advantageous because it increases the maximum depth of first liquid 1 that can be retained stably in the microfluidic arrangement without the first liquid 1 spreading laterally over the first substrate 11. This is because the weight of the first liquid 1 would tend to force the first liquid 1 downwards and therefore outwards and this effect is counteracted by buoyancy. The second liquid 2 may also advantageously increase the contact angle compared to air and so advantageously increase the volume of first liquid 1 that can be contained in a microfluidic arrangement.

In the embodiments discussed above the microfluidic arrangement is formed on an upper surface of a first substrate 11. In other embodiments, as depicted in FIG. 14, the microfluidic arrangement can be formed on a lower surface of the first substrate 11. The first substrate 11 may thus be inverted relative to the arrangement of FIG. 2. In this case, surface tension can hold the first liquid 1 in contact with the first substrate 11. The first substrate 11 and first liquid 1 can then be immersed in a bath 42 containing the second liquid 2 while the continuous body of the first liquid 1 is processed by the propelling of the separation fluid. The subsequent steps described above with reference to FIGS. 2-3 could be performed starting from the arrangement of FIG. 14. This approach may be convenient where the microfluidic arrangement is to be used for the formation of 3D cell culture spheroids for example.

In an embodiment, the continuous body of the first liquid 1 is laterally constrained predominantly by interfacial tension. For example, the continuous body of the first liquid 1 may be provided only in a selected region on the first substrate 11 rather than extending all the way to a lateral wall (e.g. where the first substrate 11 is the bottom surface of a receptacle comprising lateral walls, as depicted in FIG. 1). The continuous body is thus not laterally constrained by a lateral wall. This arrangement is particularly desirable where the second liquid 2 is denser than the first liquid 1 because it provides greater resistance against disruptions to the uniformity of thickness of the continuous body of the first liquid 1 due to downward forces on the first liquid 1 from the second liquid 2. The inventors have found that the depth of the first liquid 1 can as a consequence be higher when the first liquid 1 is laterally constrained predominantly by surface tension than when this is not the case. Providing an increased depth of the first liquid 1 is desirable because it allows larger volumes of first liquid regions for a given spatial density of features on the first substrate 11. When the microfluidic arrangement is used for culturing cells, for example, the cells may therefore be provided with higher amounts of the required materials, allowing the cells to survive longer and/or under more uniform conditions before further action needs to be taken (e.g. to supply nutrients and remove waste).

In other embodiments, the continuous body of the first liquid 1 may be allowed to extend to the lateral walls of a receptacle providing the first substrate 11. A thin film of the first liquid 1 may conveniently be formed in this way by providing a relatively deep layer of the first liquid 1 filling the bottom of the receptacle and then removing (e.g. by pipetting) the first liquid 1 to leave a thin film of the first liquid 1.

FIGS. 15 and 16 depict example apparatus 30 for performing methods according to embodiments of the present disclosure. The apparatus 30 are thus configured to manufacture a microfluidic arrangement. The apparatus 30 comprises a substrate table 16. The substrate table 16 holds a substrate 11. A continuous body of first liquid 1 is provided in direct contact with the substrate 11. A second liquid 2 is provided in direct contact with the first liquid 1. The second liquid 2 covers the first liquid 1.

A pattern forming unit is provided that propels a separation fluid 3 through the first liquid 1 and into contact with the substrate 11 over all of the selected region 4. The propulsion of the separation fluid 3 may be performed using any of the methods described above with reference to FIGS. 1-14. Alternatively or additionally, the pattern forming unit may be configured to form walls of second liquid 2 using other techniques, for example by bringing a patterned stamping member into contact with the substrate 11. The stamping member displaces the first liquid 1 to allow the second liquid 2 to form the walls of second liquid 2. The stamping member may comprise, for example, a patterned hydrophobic region to define where the second liquid 2 would be brought into contact with the substrate 11 through the first liquid 1 by the bringing into contact of the stamping member with the substrate 11.

In the example of FIG. 15, the apparatus 30 propels the separation fluid 3 by pumping the separation fluid 3 out of a distal tip 6 of an injection member 15. The apparatus 30 of FIG. 15 comprises an injection system. The injection system is configured to pump separation fluid 3 out of the distal tip 6 of the injection member 15. The injection member 15 may comprise a lumen and the separation fluid 3 may be pumped along the lumen to the distal tip 6. In an embodiment, the separation fluid 3 is ejected from the distal tip 6 while the distal tip 6 is moved over the substrate 11 according to the geometry of the selected region 4. The injection system comprises the injection member 15 and a pumping system 17. In use, the pumping system 17 will comprise a reservoir containing the separation fluid 3, conduits for conveying the separation fluid 3 from the reservoir to the lumen of the injection member 15, and a mechanism for pumping the separation fluid 3 through the lumen and out of the distal tip 6 of the injection member 15.

In an embodiment, the apparatus 30 is configured to maintain a small but finite separation between the distal tip 6 of the injection member 15 and the substrate 11 while the injection member 15 is moved over the substrate 11. This is beneficial at least where the microfluidic arrangement is to be used for cell-based studies, which would be affected by any scratching or other modification of the surface that might be caused were the injection member 15 to be dragged over the substrate 11 in contact with the substrate 11. Any such modifications could negatively affect optical access and/or cell compatibility. In an embodiment, this is achieved by mounting the injection member 15 slideably in a mounting such that a force from contact with the substrate 11 will cause the injection member 15 to slide within the mounting. Contact between the injection member 15 and the substrate 11 is detected by detecting sliding of the injection member 15 relative to the mounting. When contact is detected, the injection member 15 is pulled back by a small amount (e.g. 0.1-1 mm) before the injection member 15 is moved over the substrate 11 (without contacting the substrate 11 during this motion). This approach to controlling separation between the distal tip 6 and the substrate 11 can be implemented cost effectively in comparison to alternatives such as the capacitive/inductive methods used in 3D printers, or optical-based sensing techniques. The approach also does not require a conductive surface to be provided. In an embodiment, the separation between the distal tip 6 and the substrate 11 is varied also at later stages, after the injection member 15 has been moved some distance over the substrate 11 after the initial zeroing procedure (e.g. the initial moving back of the injection member by the small amount). For example, the formation of a wall of the second liquid 2 may be stopped (at least partly) by moving the injection member 15 further away from the substrate 11 to reduce the intensity of impingement of the separation fluid 3 or the separation might be varied to change a width of the wall of second liquid 2 being formed (moving the injection member 15 further away will generally increase a width of the wall of second liquid 2 being formed).

The injection system, or an additional injection system configured in a corresponding manner, may additionally provide the initial continuous body of the first liquid 1 in direct contact with the substrate 11 by ejecting the first liquid 1 through a distal tip of an injection member while moving the injection member over the substrate 11 to define the shape of the continuous body of the first liquid 1. In embodiments, the injection system or additional injection system may further be configured to controllably extract the first liquid 1, for example by controllably removing excess first liquid by sucking the liquid back through an injection member.

In an embodiment, the apparatus 30 comprises an application system for applying or removing the second liquid 2 (comprising for example a reservoir for holding the second liquid, an output/suction nozzle positionable above the substrate 11, and a pumping/suction mechanism for controllably pumping or sucking the second liquid 2 to/from the reservoir from/to the substrate 11 through the output/suction nozzle). In other embodiments, the second liquid 2 is applied manually.

The apparatus 30 of FIG. 15 further comprises a controller 10. The controller 10 controls movement of the injection member 15 over the substrate 11 during the propulsion of the separation fluid 3 onto the selected region on the substrate 11 (and, optionally, during forming of the continuous body of the first liquid 1). In an embodiment, the apparatus 30 comprises a processing head 20 that supports the injection member 15. The processing head 20 is configured such that the injection member 15 can be selectively advanced and retracted. In an embodiment, the advancement and retraction is controlled by the controller 10, with suitable actuation mechanisms being mounted on the processing head 20. A gantry system 21 is provided to allow the processing head 20 to move as required. In the particular example shown, left-right movement within the page is illustrated but it will be appreciated that the movement can also comprise movement into and out of the page as well as movement towards and away from the substrate 11 (if the movement of the injection member 15 provided by the processing head 20 itself is not sufficiently to provide the required upwards and downwards displacement of the injection member 15).

FIG. 16 depicts an apparatus 30 configured to propel a portion of the second liquid 2 towards the selected region by locally coupling energy into a region containing or adjacent to the portion of the second liquid 2. The apparatus of FIG. 16 may be configured to perform any of the methods described above with reference to FIGS. 9-13. The apparatus 30 comprises a laser source 22 (e.g. a sub-picosecond pulsed laser, as described above) and an optical projection system 23 configured to focus a beam provided by the laser source 22 onto a desired location. In an embodiment, the optical projection system 23 comprises a scanner for scanning a focussed laser spot along a scanning path following the geometry of the selected region 4. The scanner may be controlled by a controller 10. In an embodiment, the substrate table 16 is moved relative to the optical projection system 23 to provide, optionally in combination with scanning provided by the scanner, the scanning of the laser spot along the scanning path. The scanner may scan the spot along a first axis while the substrate table is moved along a second axis, perpendicular to the first axis, for example. Movement of the substrate table 16 may be controlled by the controller 10. Alternatively, a mask may be used to project a patterned radiation beam onto the substrate 11, a pattern of the beam corresponding to at least a portion of the selected region 4 on the substrate 11.

As mentioned in the introductory part of the description, it has been observed that alternative approaches which involve contact of a solid member with the selected region (e.g. a stylus that is scraped along the selected region to allow the second liquid to replace the first liquid along the selected region) can have a significant risk of producing walls that are discontinuous. For example, it has been observed that in arrays of sub-bodies produced using the alternative approach a small subset of the sub-bodies are found to be connected together. FIG. 17 depicts images of connections between sub-bodies of liquid (referred to as “chambers”) produced using such an alternative approach. In these particular cases, arrays of square sub-bodies (chambers) were produced, and each image shows the corners of 4 adjacent chambers with connections between some of the chambers indicated.

In the examples described above, the continuous body of the first liquid 1 and the overlying layer of second liquid 2 are provided before the separation fluid 3 is propelled through the first liquid 1 to form the walls of second liquid 2. In some embodiments, this is not the case, at least at an initial stage of the propelling of the separation fluid 3. In such embodiments, as depicted schematically in FIGS. 18 and 19, the separation fluid comprises (e.g. consists of) a liquid having the same composition as the second liquid 2. The providing of the second liquid 2 in direct contact with the continuous body of first liquid 1 and covering the continuous body of first liquid 1 comprises, after the continuous body of the first liquid 1 in direct contact with the first substrate 11 has been provided, propelling the separation fluid 3 through the first liquid 1 and into contact with the first substrate 11 along at least a portion of the selected region while a portion 50A of an upper interface of the first liquid 1 is not yet in contact with the second liquid 2. This situation is depicted in FIG. 18. The separation fluid 3 is propelled out of the distal tip 6 of an injection member and onto the selected region 4 on the first substrate 11 as indicated by the vertical arrow. Excess separation fluid 3 then moves up and outwards and starts to cover the upper interface of the first liquid 1 as indicated by the curved arrows. At the point in time depicted in FIG. 18, a portion 50B of the upper interface of the first liquid is covered by the advancing separation fluid 3 (which may also now be considered as a portion of the second liquid 2) while the portion 50A is in contact with air. The propelling of the separation fluid 3 continues until the separation fluid 3 forms a layer of second liquid 2 in direct contact with the continuous body of first liquid 1 and covering the continuous body of first liquid 1, as depicted in FIG. 19. At the stage shown in FIG. 19, no portion of the upper interface of the first liquid 1 is in contact with air. This approach is convenient because it removes the need for a user to provide the layer of second liquid as a step separate from the propelling of the separation fluid through the first liquid to form the one or more walls of second liquid. This saves time and simplifies the apparatus. Furthermore, the continuous body of the first liquid can be prepared (ready for the formation of the one or more walls of second liquid by the propelling of the separation fluid) well in advance without risk of disruption being caused by an overlaid layer of second liquid (because the layer of second liquid is not yet present). For example, prolonged overlay by the second liquid may cause variations in the depth of the first liquid prior to formation of the microfluidic arrangement with the one or more walls of second liquid, which may lead to unwanted volume variations in different regions of the microfluidic arrangement (e.g. in some sub-bodies that are isolated from each other).

In some embodiments, a separation fluid 3 is propelled through the first liquid 1 in a continuous process (i.e. without interruption) for at least a portion of the selected region 4. For example, separation fluid 3 may be propelled continuously out of a distal tip 6 of an injection member (e.g. by pumping at a continuous rate) while the distal tip 6 is moved over a portion of the selected region (e.g. in a straight line downwards as depicted in FIG. 3). In other embodiments, the propelling of the separation fluid 3 comprises intermittent propulsion of portions of the separation fluid 3 during at least a portion of the displacing of the first liquid 1 away from the selected region 4. For example, the separation fluid 3 may be propelled intermittently during the displacement of the first liquid 1 away from the selected region 4 along the portion of the selected region 4 shown in FIG. 3. In such embodiments, the intermittent propulsion may be such that the first liquid 1 is nevertheless displaced away from the selected region 4 so as to cause the selected region 4 to contact the second liquid 2 along a continuous line (e,g. as shown in FIG. 3). This may be achieved for example by arranging for different portions of the separation fluid 3 that are intermittently propelled towards the first substrate 11 (i.e. propelled at different times relative to each other) to be propelled into contact with the selected region in overlapping regions. Thus, an impact region on the first substrate 11 associated with one portion of propelled separation fluid 3 will overlap with the impact region on the first substrate 11 associated with at least one other portion of propelled separation fluid 3 (typically propelled at a slightly different time, for example after a head that is driving the propulsion has moved a short distance relative to the first substrate 11). The possibility of using intermittent propulsion opens up a wider range of possible mechanisms for driving the propulsion, such as piezoelectric mechanisms.

Further aspects of the disclosure are provided in the following numbered clauses.
1. A method of manufacturing a microfluidic arrangement, comprising:
   providing a continuous body of a first liquid in direct contact with a first substrate;
   providing a second liquid in direct contact with the continuous body of first liquid and covering the continuous body of first liquid, the second liquid being immiscible with the first liquid; and
   propelling a separation fluid, immiscible with the first liquid, through at least the first liquid and into contact with the first substrate over all of a selected region on the surface of the first substrate, thereby displacing first liquid that was initially in contact with the selected region away from the selected region without any solid member contacting the selected region directly and without any solid member contacting the selected region via a globule of liquid held at a tip of the solid member, the selected region being such that one or more walls of second liquid are formed that modify a shape of the continuous body of first liquid.
2. The method of clause 1, wherein the continuous body of first liquid remains a single continuous body of first liquid after the modification of the shape of the continuous body of first liquid by the one or more walls of second liquid.
3. The method of clause 1 or 2, wherein the separation fluid comprises one or more of the following: a gas, a liquid, a liquid having the same composition as the second liquid, and a portion of the second liquid provided before the propulsion of the separation fluid through the first liquid.
4. The method of any of clauses 1-3, wherein a wall footprint representing an area of contact between the second liquid of the wall and the first substrate of each of the one or more walls of second liquid is pinned in a static configuration by interfacial forces, the pinning being such that the wall footprint remains constant.
5. The method of clause 4, wherein an outline of the wall footprint of at least one of the walls comprises at least one straight line segment.
6. The method of clause 4, wherein an outline of the wall footprint of at least one of the walls comprises at least two non-parallel straight line segments.
7. The method of any of claims 1-6, wherein the one or more walls of second liquid define a first plurality of open-ended chambers containing the first liquid.
8. The method of clause 7, wherein the first plurality of open-ended chambers are separated from each other by the one or more walls of second liquid to the extent that there is no uninterrupted straight line path through the first liquid from the inside of any one of the open-ended chambers of the first plurality of open-ended chambers to the inside of any other one of the open-ended chambers of the first plurality of open-ended chambers.
9. The method of clause 7 or 8, wherein the one or more walls of second liquid further define one or more flow conduits configured to allow a flow of the first liquid to be driven past open ends of the first plurality of open-ended chambers.
10. The method of clause 9, wherein:
   the one or more walls of second liquid further define a second plurality of open-ended chambers, not including any of the open-ended chambers of the first plurality of open-ended chambers, the open-ended chambers of the second plurality of open-ended chambers containing the first liquid and being separated from each other by the one or more walls of second liquid to the extent that there is no uninterrupted straight line path through the first liquid from the inside of any one of the open-ended chambers of the second plurality of open-ended chambers to the inside of any other one of the open-ended chambers of the second plurality of open-ended chambers; and
   the one or more walls of second liquid define one or more flow conduits configured to allow a flow of the first liquid to be driven past open ends of the first plurality of open-ended chambers and past open ends of the second plurality of open-ended chambers.
11. The method of any of clauses 7-10, wherein at least a subset of the open-ended chambers have two open ends and the one or more walls of second liquid are configured to direct a flow of the first liquid through each of the open-ended chambers having two open ends.
12. The method of any of clauses 1-11, where the one or more walls of second liquid define at least one open-ended flow conduit.
13. The method of clause 12, wherein the open end of the open-ended flow conduit opens into a macroscopic sink volume.
14. The method of any of clauses 1-13, wherein the separation fluid is propelled onto the selected region on the first substrate by pumping the separation fluid from a distal tip of an injection member while moving the distal tip relative to the first substrate.
15. The method of clause 14, wherein the distal tip is moved through both of the second liquid and the first liquid while propelling the separation fluid onto the selected region and at least a portion of the distal tip of the injection member is configured to be more easily wetted by the second liquid than the first liquid.
16. The method of any of clauses 1-15, wherein:
   the separation fluid comprises a liquid having the same composition as the second liquid; and
   the providing of the second liquid in direct contact with the continuous body of first liquid and covering the continuous body of first liquid comprises the following, after the continuous body of the first liquid in direct contact with the first substrate has been provided:
   propelling the separation fluid through the first liquid and into contact with the first substrate in at least a portion of the selected region while a portion of an upper interface of the first liquid is not yet in contact with the second liquid, the propelling of the separation fluid continuing until the separation fluid forms a layer of second liquid in direct contact with the continuous body of first liquid and covering the continuous body of first liquid.
17. The method of any of clauses 1-15, wherein:
   the separation fluid comprises a portion of the second liquid; and
   the portion of the second liquid is propelled towards the selected region on the first substrate by locally coupling energy into a region containing or adjacent to the portion of the second liquid to be propelled towards the selected region on the first substrate.
18. The method of clause 17, wherein the local coupling of energy is achieved using a focussed beam of electromagnetic radiation or ultrasound.
19. The method of clause 18, wherein a focus of the beam is scanned along a scanning path based on the geometry of the selected region.
20. The method of clause 18 or 19, wherein:
   the first substrate comprises a first base layer and a first intermediate absorbing layer between the first base layer and the first liquid;
   a beam absorbance per unit thickness of the first intermediate absorbing layer is higher than a beam absorbance per unit thickness of the first base layer; and
   energy from the beam absorbed in the first intermediate absorbing layer causes the first liquid to be locally forced away from the first substrate in the selected region, the second liquid moving into contact with the first substrate where the first liquid has been forced away.
21. The method of clause 18 or 19, further comprising a second substrate facing at least a portion of the first substrate and in contact with liquid, such that there is a continuous liquid path between the second substrate and the first substrate.
22. The method of clause 21, wherein energy from the beam absorbed in either or both of the second substrate and liquid adjacent to the second substrate causes the second liquid to be locally forced away from the second substrate, thereby providing the propulsion of the second liquid towards the selected region on the first substrate.
23. The method of clause 21 or 22, wherein:
   the second substrate comprises a second base layer and a second intermediate absorbing layer between the second base layer and the second liquid;
   a beam absorbance per unit thickness of the second intermediate absorbing layer is higher than a beam absorbance per unit thickness of the second base layer; and
   energy from the beam absorbed in the second intermediate absorbing layer causes the second liquid to be locally forced away from the second substrate, thereby providing the propulsion of the second liquid towards the selected region on the first substrate.
24. The method of any of clauses 18-23, wherein:
   a layer of a third liquid is provided above the second liquid;
   a beam absorbance per unit thickness of the third liquid is higher than a beam absorbance per unit thickness of the second liquid; and
   energy from the beam absorbed in the third liquid causes the second liquid to be locally propelled towards the selected region on the first substrate.
25. A method of operating a microfluidic arrangement, comprising:
   providing a microfluidic arrangement comprising a continuous body of a first liquid in direct contact with a substrate, and a second liquid in direct contact with the continuous body of first liquid and covering the continuous body of first liquid, the second liquid being immiscible with the first liquid, wherein one or more walls of second liquid are pinned in contact with a selected region of the substrate to define a shape of the continuous body of first liquid, wherein:
   the one or more walls of second liquid define a plurality of open-ended chambers containing the first liquid; and
   the method further comprises:
   providing target material different from the first liquid and the second liquid in each of a plurality of the open-ended chambers; and
   driving a flow of the first liquid past open ends of the open-ended chambers or through the open-ended chambers.
26. The method of clause 25, wherein the target material comprises biological material.
27. The method of clause 25 or 26, wherein the target material is provided in the continuous body of first liquid before the one or more walls of second liquid are formed.
28. An apparatus for manufacturing a microfluidic arrangement, comprising:
   a substrate table configured to hold a substrate on which a continuous body of a first liquid is provided in direct contact with a substrate, and a second liquid is provided in direct contact with the first liquid and covering the first liquid, the second liquid being immiscible with the first liquid; and
   a pattern forming unit configured to propel a separation fluid, immiscible with the first liquid, through at least the first liquid and into contact with the first substrate over all of a selected region on the surface of the first substrate, thereby displacing first liquid that was initially in contact with the selected region away from the selected region without any solid member contacting the selected region directly and without any solid member contacting the selected region via a globule of liquid held at a tip of the solid member, the selected region being such that one or more walls of second liquid are formed that modify a shape of the continuous body of first liquid.

1. A method of manufacturing a microfluidic arrangement, comprising:
providing a continuous body of a first liquid in direct contact with a first substrate;
providing a second liquid in direct contact with the continuous body of first liquid and covering the continuous body of first liquid, the second liquid being immiscible with the first liquid; and
propelling a separation fluid, immiscible with the first liquid, through at least the first liquid and into contact with the first substrate over all of a selected region on the surface of the first substrate, thereby displacing first liquid that was initially in contact with the selected region away from the selected region without any solid member contacting the selected region directly and without any solid member contacting the selected region via a globule of liquid held at a tip of the solid member, the selected region being such that one or more walls of second liquid are formed that modify a shape of the continuous body of first liquid.

2. The method of claim 1, wherein the continuous body of first liquid remains a single continuous body of first liquid after the modification of the shape of the continuous body of first liquid by the one or more walls of second liquid.

3. The method of claim 1, wherein the separation fluid comprises one or more of the following: a gas, a liquid, a liquid having the same composition as the second liquid, and a portion of the second liquid provided before the propulsion of the separation fluid through the first liquid.

4. The method of claim 1, wherein a wall footprint representing an area of contact between the second liquid of the wall and the first substrate of each of the one or more walls of second liquid is pinned in a static configuration by interfacial forces, the pinning being such that the wall footprint remains constant.

5. The method of claim 4, wherein an outline of the wall footprint of at least one of the walls comprises at least one straight line segment.

6. The method of claim 4, wherein an outline of the wall footprint of at least one of the walls comprises at least two non-parallel straight line segments.

7. The method of claim 1, wherein the one or more walls of second liquid define a first plurality of open-ended chambers containing the first liquid.

8. The method of claim 7, wherein the first plurality of open-ended chambers are separated from each other by the one or more walls of second liquid to the extent that there is no uninterrupted straight line path through the first liquid from the inside of any one of the open-ended chambers of the first plurality of open-ended chambers to the inside of any other one of the open-ended chambers of the first plurality of open-ended chambers.

9. The method of claim 7, wherein the one or more walls of second liquid further define one or more flow conduits configured to allow a flow of the first liquid to be driven past open ends of the first plurality of open-ended chambers.

10. The method of claim 9, wherein:
the one or more walls of second liquid further define a second plurality of open-ended chambers, not including any of the open-ended chambers of the first plurality of open-ended chambers, the open-ended chambers of the second plurality of open-ended chambers containing the first liquid and being separated from each other by the one or more walls of second liquid to the extent that there is no uninterrupted straight line path through the first liquid from the inside of any one of the open-ended chambers of the second plurality of open-ended chambers to the inside of any other one of the open-ended chambers of the second plurality of open-ended chambers; and
the one or more walls of second liquid define one or more flow conduits configured to allow a flow of the first liquid to be driven past open ends of the first plurality of open-ended chambers and past open ends of the second plurality of open-ended chambers.

11. The method of claim 7, wherein at least a subset of the open-ended chambers have two open ends and the one or more walls of second liquid are configured to direct a flow of the first liquid through each of the open-ended chambers having two open ends.

12. The method of claim 1, where the one or more walls of second liquid define at least one open-ended flow conduit.

13. The method of claim 12, wherein the open end of the open-ended flow conduit opens into a macroscopic sink volume.

14. The method of claim 1, wherein the separation fluid is propelled onto the selected region on the first substrate by pumping the separation fluid from a distal tip of an injection member while moving the distal tip relative to the first substrate.

15. The method of claim 14, wherein the distal tip is moved through both of the second liquid and the first liquid while propelling the separation fluid onto the selected region and at least a portion of the distal tip of the injection member is configured to be more easily wetted by the second liquid than the first liquid.

16. The method of claim 1, wherein:
the separation fluid comprises a liquid having the same composition as the second liquid; and
the providing of the second liquid in direct contact with the continuous body of first liquid and covering the continuous body of first liquid comprises the following, after the continuous body of the first liquid in direct contact with the first substrate has been provided:
propelling the separation fluid through the first liquid and into contact with the first substrate in at least a portion of the selected region while a portion of an upper interface of the first liquid is not yet in contact with the second liquid, the propelling of the separation fluid continuing until the separation fluid forms a layer of second liquid in direct contact with the continuous body of first liquid and covering the continuous body of first liquid.

17. The method of claim 1, wherein:
the separation fluid comprises a portion of the second liquid; and
the portion of the second liquid is propelled towards the selected region on the first substrate by locally coupling energy into a region containing or adjacent to the portion of the second liquid to be propelled towards the selected region on the first substrate.

18. The method of claim 17, wherein the local coupling of energy is achieved using a focussed beam of electromagnetic radiation or ultrasound.

19. The method of claim 18, wherein a focus of the beam is scanned along a scanning path based on the geometry of the selected region.

20. The method of claim 18, wherein:
the first substrate comprises a first base layer and a first intermediate absorbing layer between the first base layer and the first liquid;
a beam absorbance per unit thickness of the first intermediate absorbing layer is higher than a beam absorbance per unit thickness of the first base layer; and
energy from the beam absorbed in the first intermediate absorbing layer causes the first liquid to be locally forced away from the first substrate in the selected region, the second liquid moving into contact with the first substrate where the first liquid has been forced away.

21. The method of claim 18, further comprising a second substrate facing at least a portion of the first substrate and in contact with liquid, such that there is a continuous liquid path between the second substrate and the first substrate.

22. The method of claim 21, wherein energy from the beam absorbed in either or both of the second substrate and liquid adjacent to the second substrate causes the second liquid to be locally forced away from the second substrate, thereby providing the propulsion of the second liquid towards the selected region on the first substrate.

23. The method of claim 21, wherein:
the second substrate comprises a second base layer and a second intermediate absorbing layer between the second base layer and the second liquid;
a beam absorbance per unit thickness of the second intermediate absorbing layer is higher than a beam absorbance per unit thickness of the second base layer; and
energy from the beam absorbed in the second intermediate absorbing layer causes the second liquid to be locally forced away from the second substrate, thereby providing the propulsion of the second liquid towards the selected region on the first substrate.

24. The method of claim 18, wherein:
a layer of a third liquid is provided above the second liquid;
a beam absorbance per unit thickness of the third liquid is higher than a beam absorbance per unit thickness of the second liquid; and
energy from the beam absorbed in the third liquid causes the second liquid to be locally propelled towards the selected region on the first substrate.

25. A method of operating a microfluidic arrangement, comprising:
providing a microfluidic arrangement comprising a continuous body of a first liquid in direct contact with a substrate, and a second liquid in direct contact with the continuous body of first liquid and covering the continuous body of first liquid, the second liquid being immiscible with the first liquid, wherein one or more walls of second liquid are pinned in contact with a selected region of the substrate to define a shape of the continuous body of first liquid, wherein:
the one or more walls of second liquid define a plurality of open-ended chambers containing the first liquid; and
the method further comprises:
providing target material different from the first liquid and the second liquid in each of a plurality of the open-ended chambers; and
driving a flow of the first liquid past open ends of the open-ended chambers or through the open-ended chambers.

26. The method of claim 25, wherein the target material comprises biological material.

27. The method of claim 25, wherein the target material is provided in the continuous body of first liquid before the one or more walls of second liquid are formed.

28. An apparatus for manufacturing a microfluidic arrangement, comprising:
a substrate table configured to hold a substrate on which a continuous body of a first liquid is provided in direct contact with a substrate, and a second liquid is provided in direct contact with the first liquid and covering the first liquid, the second liquid being immiscible with the first liquid; and
a pattern forming unit configured to propel a separation fluid, immiscible with the first liquid, through at least the first liquid and into contact with the first substrate over all of a selected region on the surface of the first substrate, thereby displacing first liquid that was initially in contact with the selected region away from the selected region without any solid member contacting the selected region directly and without any solid member contacting the selected region via a globule of liquid held at a tip of the solid member, the selected region being such that one or more walls of second liquid are formed that modify a shape of the continuous body of first liquid.
